(19) United States
(12) Patent Application Publication
(10) Pub. No.: US 2020/0205781 A1
(43) Pub. Date: Jul. 2, 2020

(54) Miniscule Transducer for a Medical Article

(71) Applicant: Avent, Inc., Alpharetta, GA (US)

(72) Inventors: Steve S. Khalaj, Laguna Hills, CA (US); Marc Comtois, Irvine, CA (US); Tingting Wang, Irvine, CA (US); Shirzad Shahriari, Irvine, CA (US); Shiva Sharareh, Laguna Niguel, CA (US)

(21) Appl. No.: 16/233,651

(22) Filed: Dec. 27, 2018

(51) Int. Cl. A61B 8/00; A61B 8/08

(52) U.S. Cl. CPC A61B 8/4483; A61B 8/445; A61B 8/12; A61B 8/461; A61B 8/5223; A61B 8/0841

(57) ABSTRACT

An active needle assembly for use with an ultrasound imaging system includes a needle having a proximal end and a distal end. The distal end is adapted to be inserted into a patient. The needle assembly also includes a needle transducer mounted to an exterior surface of the needle. Further, the needle transducer has an area that is less than about two (2) square millimeters (mm²). Moreover, the needle assembly includes an electrical connection for connecting the needle transducer to a power source.

### FIELD OF THE INVENTION

The present invention relates generally to miniscule transducers for medical articles, such as needles, catheters, and/or stylets, for use with autonomous ultrasound imaging systems.

### BACKGROUND

Detection of anatomical objects using medical imaging is an essential step for many medical procedures, such as regional anesthesia nerve blocks, and is becoming the standard in clinical practice to support diagnosis, patient stratification, therapy planning, intervention, and/or follow-up. Various systems based on traditional approaches exist for anatomical detection and tracking in medical images, such as computed tomography (CT), magnetic resonance (MR), ultrasound, and fluoroscopic images.

For example, ultrasound imaging systems utilize sound waves with frequencies higher than the upper audible limit of human hearing. Further, ultrasound imaging systems are widely used in medicine to perform both diagnosis and therapeutic procedures. In such procedures, sonographers perform scans of a patient using a hand-held probe or transducer that is placed directly on and moved over the patient.

Certain ultrasound systems may be used in combination with needles having active (i.e. electrically-powered) transducers, which require an electrical connection to a power source. Such needle assemblies typically route cabling from the power source through a lumen of the needle and to the transducer. For conventional assemblies, the transducers are required to be large enough to maintain signal purity and/or fidelity. In certain instances, however, signal purity may be irrelevant or insignificant.

Accordingly, the present disclosure is directed to a needle assembly having a miniscule transducer mounted thereto that can be used when signal purity and/or fidelity is not of concern.

### SUMMARY OF THE INVENTION

Objects and advantages of the invention will be set forth in part in the following description, or may be obvious from the description, or may be learned through practice of the invention.

In one aspect, the present invention is directed to an active needle assembly for use with an ultrasound imaging system. The needle assembly includes a needle having a proximal end and a distal end. The distal end is adapted to be inserted into a patient. The needle assembly also includes a needle transducer mounted to an exterior surface of the needle. Further, the needle transducer has an area that is less than about two (2) square millimeters (mm²), more preferably about 1.5 mm², or up to about 0.6 mm², or up to about 0.2 mm². Moreover, the needle assembly includes an electrical connection for connecting the needle transducer to a power source.

In one embodiment, the needle transducer has a width of up to about one (1) mm, more preferably up to about 0.5 mm, and still more preferably up to about 0.2 mm. In another embodiment, the needle transducer has a length of up to about one (1) mm, more preferably up to about 0.5 mm. Thus, in certain embodiments, the area of the needle transducer may be a square or a rectangular shape.

In further embodiments, the needle transducer may be mounted to the exterior surface of the needle via bonding, or an additive manufacturing process. In additional embodiments, the needle transducer may be mounted at the distal end of the needle. In another embodiment, the needle transducer may be a single transducer or multiple transducers arranged in an array.

In yet another embodiment, the electrical connection of the needle assembly may include a flexible printed circuit board and/or one or more cables.

In another aspect, the present disclosure is directed to an active transducer assembly for use with an ultrasound imaging system. The transducer assembly includes an article having a proximal end and a distal end. The distal end is adapted to be inserted into a patient. The transducer assembly also includes an article transducer mounted to an exterior surface of the article. Further, the article transducer has an area less than about 4 square millimeters (mm²). Moreover, the transducer assembly includes an electrical connection for connecting the article transducer to a power source. In one embodiment, the article may be a needle, a catheter, or a stylet. In addition, it should be understood that the transducer assembly may further include any of the additional features described herein.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of one embodiment of an imaging system according to the present disclosure;

FIG. 2 illustrates a block diagram one of embodiment of a controller of an imaging system according to the present disclosure;

FIG. 3 illustrates a schematic diagram of one embodiment of a needle assembly according to the present disclosure;

FIG. 4 illustrates a schematic diagram of one embodiment of a catheter according to the present disclosure;

FIG. 5 illustrates a schematic diagram of one embodiment of a stylet according to the present disclosure;

FIG. 6 illustrates a top view of one embodiment of a miniscule article transducer according to the present disclosure;

FIG. 7A illustrates a perspective view of one embodiment of a distal end of a needle assembly according to the present disclosure, particularly illustrating the location for a transducer and corresponding wire on an embedded flat portion on the needle;

FIG. 7B illustrates a cross-sectional view of the needle assembly of FIG. 7A; and

FIG. 8 illustrates a perspective view of one embodiment of the needle assembly according to the present disclosure, particularly illustrated the article transducer mounted on the needle.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

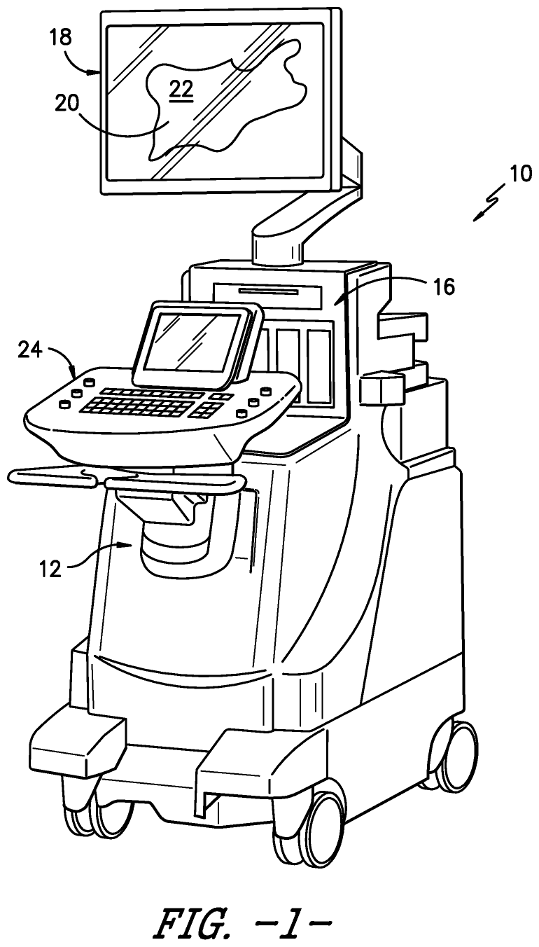

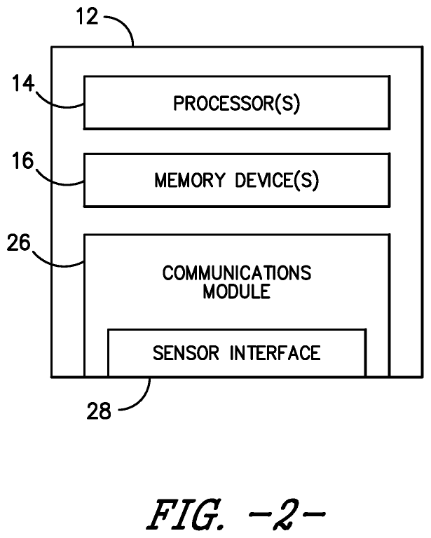

Referring now to the drawings, FIGS. 1 and 2 illustrate a medical imaging system 10 for scanning, identifying, and navigating anatomical objects of a patient according to the present disclosure. As used herein, the anatomical object(s) 22 and surrounding tissue described herein may include any anatomical structure and/or surrounding tissue of a patient. For example, in one embodiment, the anatomical object(s) 22 may include one or more nerves or nerve bundles. More specifically, in another embodiment, the anatomical object(s) 22 may include an interscalene brachial plexus of the patient, which generally corresponds to the network of nerves running from the spine, formed by the anterior rami of the lower four cervical nerves and first thoracic nerve. As such, the surrounding tissue of the brachial plexus generally corresponds to the sternocleidomastoid muscle, the middle scalene muscle, the anterior scalene muscle, and/or similar.

It should be understood, however, that the system of the present disclosure may be further used for any variety of medical procedures involving any anatomical structure in addition to those relating to the brachial plexus. For example, the anatomical object(s) 22 may include upper and lower extremities, as well as compartment blocks. More specifically, in such embodiments, the anatomical object(s) 22 of the upper extremities may include interscalene muscle, supraclavicular muscle, infraclavicular muscle, and/or axillary muscle nerve blocks, which all block the brachial plexus (a bundle of nerves to the upper extremity), but at different locations. Further, the anatomical object(s) 22 of the lower extremities may include the lumbar plexus, the fascia Iliac, the femoral nerve, the sciatic nerve, the abductor canal, the popliteal, the saphenous (ankle), and/or similar. In addition, the anatomical object(s) 22 of the compartment blocks may include the intercostal space, transversus abdominis plane, and thoracic paravertebral space, and/or similar.

In addition, as shown, the imaging system 10 may correspond to an ultrasound imaging system or any other suitable imaging system that can benefit from the present technology. Thus, as shown, the imaging system 10 may generally include a controller 12 having one or more processor(s) 14 and associated memory device(s) 16 configured to perform a variety of computer-implemented functions (e.g., performing the methods and the like and storing relevant data as disclosed herein), as well as a user display 18 configured to display an image 20 of an anatomical object 22 or the surrounding tissue to an operator. In addition, the imaging system 10 may include a user interface 24, such as a computer and/or keyboard, configured to assist a user in generating and/or manipulating the user display 18.

Additionally, as shown in FIG. 2, the processor(s) 14 may also include a communications module 26 to facilitate communications between the processor(s) 14 and the various components of the imaging system 10, e.g. any of the components of FIG. 1. Further, the communications module 26 may include a sensor interface 28 (e.g., one or more analog-to-digital converters) to permit signals transmitted from one or more probes (e.g. such as an ultrasound transducer and/or an article transducer 30 as described herein) to be converted into signals that can be understood and processed by the processor(s) 14.

It should be appreciated that the various probes and/or transducers described herein may be communicatively coupled to the communications module 26 of the controller 12 using any suitable means. For example, as shown in FIG. 2, the article transducer 30 may be coupled to the sensor interface 28 via a wired connection. However, in other embodiments, the article transducer 30 may be coupled to the sensor interface 28 via a wireless connection, such as by using any suitable wireless communications protocol known in the art. As such, the processor(s) 14 may be configured to receive one or more sensor signals from the article transducer 30.

As used herein, the term “processor” refers not only to integrated circuits referred to in the art as being included in a computer, but also refers to a controller, a microcontroller, a microcomputer, a programmable logic controller (PLC), an application specific integrated circuit, a field-programmable gate array (FPGA), an Application-Specific Integrated Circuit (ASIC), and other programmable circuits. The processor(s) 14 is also configured to compute advanced control algorithms and communicate to a variety of Ethernet or serial-based protocols (Modbus, OPC, CAN, etc.). Furthermore, in certain embodiments, the processor(s) 14 may communicate with a server through the Internet for cloud computing in order to reduce the computation time and burden on the local device. Additionally, the memory device(s) 16 may generally comprise memory element(s) including, but not limited to, computer readable medium (e.g., random access memory (RAM)), computer readable non-volatile medium (e.g., a flash memory), a floppy disk, a compact disc-read only memory (CD-ROM), a magneto-optical disk (MOD), a digital versatile disc (DVD) and/or other suitable memory elements. Such memory device(s) 16 may generally be configured to store suitable computer-readable instructions that, when implemented by the processor(s) 14, configure the processor(s) 14 to perform the various functions as described herein.

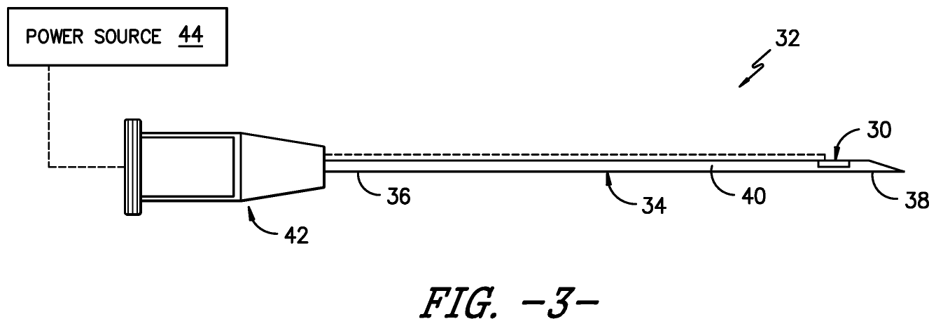

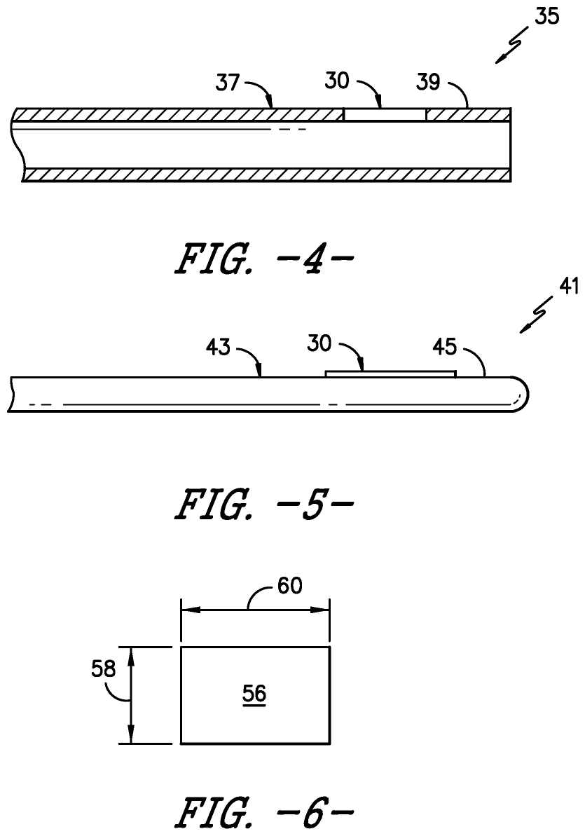

Referring now to FIGS. 3-5, various views of embodiments of the present disclosure are provided to illustrate the miniscule transducers mounted to various medical articles. For example, in certain embodiments, the medical articles described herein may include needles, catheters, stylets, or similar. Accordingly, FIG. 3 illustrates a side view of one embodiment of a needle assembly 32 for use with the ultrasound imaging system 10 according to the present disclosure. FIG. 4 illustrates a side view of one embodiment of a catheter 35 for use with the ultrasound imaging system 10 according to the present disclosure. FIG. 5 illustrates a side view of one embodiment of a stylet 41 for use with the ultrasound imaging system 10 according to the present disclosure.

Referring particularly to FIG. 3, the needle assembly 32 includes a needle 34 having a proximal end 36 and a distal end 38 adapted to be inserted into a patient. Further, as shown, the needle assembly 32 includes the article transducer 30, which may be mounted to an exterior surface 40 of the needle 34 at the distal end 38 thereof. It should be understood, however, that the article transducer 30 may be mounted at any suitable location on the needle 34. In addition, in alternative embodiments, as shown in FIG. 4, the article transducer 30 may be mounted to an exterior surface 37 of the catheter 35, e.g. at a distal end 39 thereof. It should be understood, however, that the article transducer 30 may be mounted at any suitable location on the catheter 35. In still another embodiment, as shown in FIG. 5, the article transducer 30 may be mounted to an exterior surface 43 of the stylet 41, e.g. at the distal end 45 thereof. It should be understood, however, that the article transducer 30 may be mounted at any suitable location on the stylet 41.

In addition, as shown in FIG. 6, the article transducer 30 of the present disclosure may have an area 56 that is less than about two (2) square millimeters (mm²). More specifically, as shown, the article transducer 30 may have a width 58 of up to about one (1) mm, more preferably up to about 0.5 mm, and still more preferably up to about 0.2 mm. In addition, as shown, the article transducer 30 may have a length 60 of up to about one (1) mm, more preferably up to about 0.5 mm. Thus, as shown in the illustrated embodiment, the area 56 of the article transducer 30 may be a square or a rectangular shape. The small size of the article transducer 30 is possible due to the use of the transducer with respect to the ultrasound imaging system 10. For example, the article transducer 30 of the present disclosure is configured to generate a location signal that can be picked up by the ultrasound imaging system 10, but is not needed for imaging.

For example, in a conventional PZT transducer, the overall thickness can be reduced by eliminating most, if not all, of its backing layer. This causes the PZT transducer to ring, which is of no consequence in ultrasound applications. In imaging, the backing, or damping element, reduces ringing and dampens the sound pulse. These parameters are specified for imaging applications because they benefit from reduced ringing to improve resolution. Certain PZT implementations use alternatives that reduce device thickness by design. There is no backing to eliminate in such a device and its performance remains suitable for imaging applications, same as CMUTs. Thus, the small size of the transducer is possible because the backing thereof can be eliminated, but also because the transmit and receive bands of the transducer 30 of the present disclosure do not need to be particularly well-behaved. For instance, the transducer 30 of the present disclosure can afford passband ripples of several dB. As such, the passband of the transducer 30 can be defined at lower cut-off levels than the usual 3 dB for imaging applications, e.g. at about 10 dB or even 20 dB.

The article transducer 30 will now be discussed in reference to the needle assembly 32 of FIG. 3, however, it should be understood that any of the features illustrated in FIG. 3 may also be combined with the catheter 35 and/or stylet 41 embodiments of FIGS. 4 and 5. Referring back to FIG. 3, the needle 34 may also include a needle hub 42 at its proximal end 36. In such embodiments, the article transducer(s) 30 may be communicatively coupled to a power source 44 via the needle hub 42 that provides electrical power to the transducer(s) 30. In certain embodiments, the power source 44 may be part of the ultrasound imaging system 10 or may be separate component such that the needle assembly 32 is completely autonomous from the ultrasound imaging system 10.

In addition, the article transducer 30 may be mounted to the exterior surface of the needle 34, catheter 35, and/or stylet 41 via any suitable process, such as bonding, or an additive manufacturing process. Further, the article transducer(s) 30 may be any suitable transducer now known or later developed in the art. For example, in one embodiment, the transducer(s) 30 may be a piezoelectric (PZT) transducer. Alternatively, the transducer(s) 30 may be a capacitive micromachined ultrasonic (CMUT) transducer. In yet another embodiment, the transducer(s) 30 may also include Polydimethylsiloxane (PDMS) transducers and/or photoacoustic transducers.

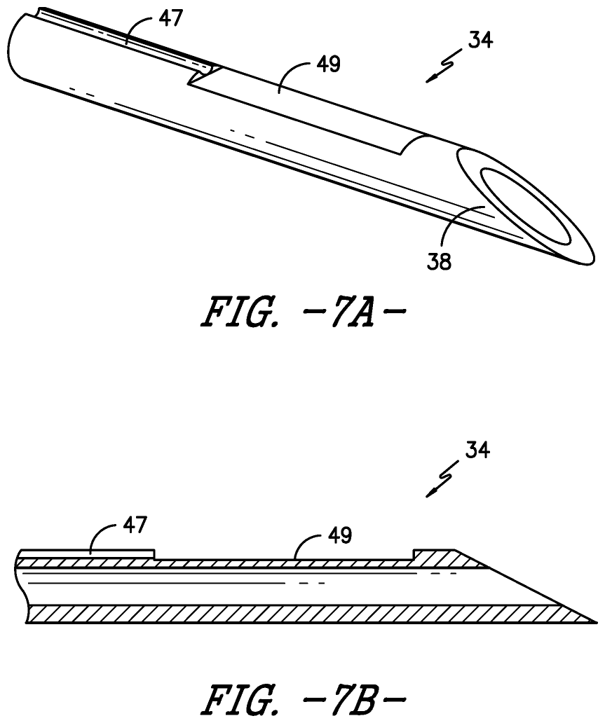

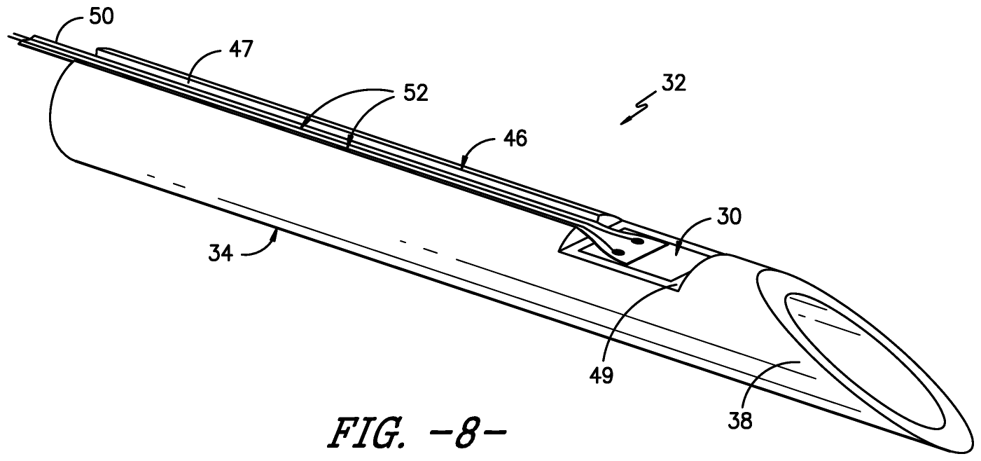

Referring particularly to FIGS. 7A and 7B, various views of one embodiment of the needle 34 of the needle assembly 32 are illustrated. More specifically, FIGS. 7A and 7B illustrate a perspective view and a cross-sectional view, respectively, of one embodiment of the distal end 38 of the needle 34 according to the present disclosure. As shown, the illustrated embodiment depicts a location for one of the article transducers 30 and corresponding traces 52 or wires within an embedded flat portion 49 within the needle wall and a recess extending therefrom. Thus, as shown, the front portion of the needle wall (which allows the article transducer 30 to be embedded therein) is configured to protect the article transducer 30 at the time of insertion within a patient. In addition, in such embodiments, the traces 52 are configured to provide the electrical connection for connecting the article transducer 30 to the power source 44. In another embodiment, the electrical connection of the needle assembly 32 may include one or more cables connected between the transducer 30 and the power source 44, e.g. within or outside of the needle 34.

Referring now to FIG. 8, a perspective view of one embodiment of the needle assembly 32 having the article transducer 30 mounted on the needle 34 is illustrated. As shown, the article transducer 30 may be arranged within the flat portion 49 of the needle 34. In addition, as shown, the transducer 30 may be electrically connected to the power source 44 via a flexible printed circuit board 46. For example, as shown, the flexible circuit board 46 may include a flexible base 50 having one or more conductive tracks 52 or traces printed thereon. As such, the flexible base 50 can easily flex with the shape of the needle 34 so as to be effectively mounted onto the exterior surface 40 of needle 34.

For example, in certain embodiments, the conductive tracks or traces 52 may be printed onto the flexible base 50 via screen printing, flexography, gravure printing, offset lithography, inkjet printing, additive manufacturing, or any other suitable printing process. In addition, in such embodiments, the conductive tracks 52 may be narrow, such as from about 0.10 millimeter (mm) up to about 0.25 mm. Further, in certain embodiments, ground planes can be used to enclose the signal trace to achieve better noise immunity. In addition, as shown, the plurality of conductive tracks 52 may include a first conductive track configured to send signals from the article transducer 30 and a second conductive track configured to receive signals from the ultrasound imaging system 10. In addition, as shown in the illustrated embodiment, the recess 47 of the needle 34 may be configured to receive the flexible base 50 containing the conductive traces 52.

In additional embodiments, the conductive traces 52 may include a single core wire, a coaxial cable, or any other suitable cable or wire. For example, in one embodiment, the conductive traces 52 may include a solid- or multi-strand wire, such as an insulated wire of a small gauge (e.g. in the order of 40 AWG or smaller). In another embodiment, the conductive traces 52 may include a coaxial cable of a small gauge (e.g. in the order of 40 AWG or smaller) so as to provide a better noise immunity environment.

In certain embodiments, the additive manufacturing process described herein may include, for example, of directed energy deposition, direct laser deposition, or any other suitable additive manufacturing technique. By using additive manufacturing, the conductive traces 52 can be printed onto the flexible circuit board 46 or directly onto the needle 34 in thin layers so as not to disturb the overall efficacy of the needle 34 in puncturing the necessary tissue of the patient. For example, in one embodiment, each of the conductive traces 52 may have a predetermined thickness ranging from about 0.01 millimeters (mm) to about 0.05 mm. As used herein, terms of degree, such as “about,” are meant to encompass a range of +/−10% from the value set forth.

It should also be understood that interconnection of the various electrical connections described herein (e.g. the flexible printed circuit board 46) and the article transducer 30 can be achieved via a variety of methods. For example, in certain embodiments, the various electrical connections may be made via soldering and/ or by using a conductive or non-conductive epoxy joint, i.e. with or without a polychlorinated biphenyl (PCB) interface, which can be used to wire bond to the device rather than connecting directly to the wire/cable.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. An active needle assembly for use with an ultrasound imaging system, the needle assembly comprising:
a needle comprising a proximal end and a distal end, the distal end adapted to be inserted into a patient;
a needle transducer mounted to an exterior surface of the needle, the needle transducer comprising an area less than about two (2) square millimeters (mm²); and
an electrical connection for connecting the needle transducer to a power source.

2. The needle assembly of claim 1, wherein the needle transducer comprises a width of up to about one (1) mm.

3. The needle assembly of claim 2, wherein the width is up to about 0.5 mm.

4. The needle assembly of claim 3, wherein the width is up to about 0.2 mm.

5. The needle assembly of claim 1, wherein the needle transducer comprises a length of up to about one (1) mm.

6. The needle assembly of claim 5, wherein the length is up to about 0.5 mm.

7. The needle assembly of claim 1, wherein the area of the needle transducer comprises at least one a square or a rectangular shape.

8. The needle assembly of claim 1, wherein the needle transducer is mounted to the exterior surface of the needle via at least one of bonding or an additive manufacturing process.

9. The needle assembly of claim 1, wherein the needle transducer is mounted at the distal end of the needle.

10. The needle assembly of claim 1, wherein the electrical connection comprises at least one of a flexible printed circuit board or one or more cables.

11. An active transducer assembly for use with an ultrasound imaging system, the transducer assembly comprising:
an article comprising a proximal end and a distal end, the distal end adapted to be inserted into a patient;
an article transducer mounted to an exterior surface of the article, the article transducer comprising an area less than about two (2) square millimeters (mm²); and
an electrical connection for connecting the article transducer to a power source.

12. The transducer assembly of claim 10, wherein the article comprises at least one of a needle, a catheter, or a stylet.

13. The transducer assembly of claim 10, wherein the needle transducer comprises a width of up to about one (1) mm.

14. The transducer assembly of claim 13, wherein the width is up to about 0.5 mm.

15. The transducer assembly of claim 14, wherein the width is up to about 0.2 mm.

16. The transducer assembly of claim 10, wherein the needle transducer comprises a length of up to about one (1) mm.

17. The transducer assembly of claim 10, wherein the area of the needle transducer comprises at least one a square or a rectangular shape.

18. The transducer assembly of claim 10, wherein the needle transducer is mounted to the exterior surface of the needle via at least one of bonding or an additive manufacturing process.

19. The transducer assembly of claim 10, wherein the needle transducer is mounted at the distal end of the needle.

20. The transducer assembly of claim 10, wherein the electrical connection comprises at least one of a flexible printed circuit board or one or more cables.
